(19) United States
(12) Patent Application Publication
(10) Pub. No.: US 2015/0088123 A1
(43) Pub. Date: Mar. 26, 2015

(54) LIQUID EJECTING APPARATUS FOR MEDICAL TREATMENT

(71) Applicant: SEIKO EPSON CORPORATION, Tokyo (JP)

(72) Inventors: Hirokazu SEKINO, Chino-shi (JP); Koichiro MIYAZAKI, Ina-shi (JP)

(21) Appl. No.: 14/491,748

(22) Filed: Sep. 19, 2014

(30) Foreign Application Priority Data
Sep. 20, 2013 (JP) 2013-194758

(51) Int. Cl. A61B 17/3203; A61B 18/14; A61B 18/12; A61B 17/32

(52) U.S. Cl. CPC A61B 17/3203; A61B 17/320068; A61B 18/14; A61B 18/1206; A61B 2018/00601
USPC 606/39; 606/169

(57) ABSTRACT

A liquid ejecting apparatus for medical treatment includes a liquid ejecting scalpel that ejects a liquid, an ultrasonic scalpel that generates an ultrasonic wave, an inner body that accommodates the liquid ejecting scalpel and the ultrasonic scalpel and includes a first opening portion allowing a distal end portion of the liquid ejecting scalpel or a distal end portion of the ultrasonic scalpel to protrude therethrough, an outer body that accommodates the inner body and includes a second opening portion at a position corresponding to the first opening portion, a suctioning path that is made by an aperture formed between the inner body and the outer body, and a manipulation section that is arranged in the outer body and selectively causes the distal end portion of the liquid ejecting scalpel or the distal end portion of the ultrasonic scalpel to protrude through the first opening portion.

This patent application claims the benefit of Japanese Patent Application No. 2013-194758, filed on Sep. 20, 2013. The content of the aforementioned application is hereby incorporated by reference in its entirety.

### BACKGROUND

1. Technical Field

The present invention relates to a technology of a liquid ejecting apparatus for medical treatment.

2. Related Art

As surgical instruments which are used to carry out incision, excision, and the like of a biological tissue, there are known a liquid ejecting-type liquid ejecting scalpel, an electrical scalpel, an ultrasonic scalpel, and a laser scalpel (for example, JP-A-5-92009 described below).

Incidentally, medical doctors use appropriately different surgical instruments in order to carry out incision, excision, and hemostasis of a biological tissue among those surgical instruments, and thus, there has been a disadvantage that the operation has to be interrupted to switch the surgical instrument.

For example, in a case of a neurosurgical procedure, a medical doctor performs a surgical operation by using the surgical instruments while magnifying a lesion site by a microscope and the like. In the related art, medical doctors have to refocus their attention on the distal end of the surgical instrument every time they switch the surgical instrument, and thus, there has been a disadvantage that the repeated switching leads to poor concentration and physical exhaustion.

In a case of endoscopic/laparoscopic surgery, in order to replace the surgical instrument to be used, a medical doctor needs to draw the endoscope/laparoscope out of the body of a patient and to switch the distal end of the surgical instrument outside the body of the patient, thereby reinserting the endoscope/laparoscope into the patient. A series of the operations causes a disadvantage of deterioration in efficiency of the surgical operation.

For example, when employing a structure in which multiple types of scalpels are integrated, there is a disadvantage that the structure increases in size.

### SUMMARY

An advantage of some aspects of the invention is to solve at least a part of the problems described above, and the invention can be implemented as the following forms.

(1) An aspect of the invention provides a liquid ejecting apparatus for medical treatment. The liquid ejecting apparatus for medical treatment includes a liquid ejecting scalpel that ejects a liquid, an ultrasonic scalpel that generates an ultrasonic wave, an inner body that accommodates the liquid ejecting scalpel and the ultrasonic scalpel and includes a first opening portion allowing a distal end portion of the liquid ejecting scalpel or a distal end portion of the ultrasonic scalpel to protrude therethrough, an outer body that accommodates the inner body and includes a second opening portion at a position corresponding to the first opening portion, a suctioning path that is made by an aperture formed between the inner body and the outer body, and a manipulation section that is arranged in the outer body and selectively causes the distal end portion of the liquid ejecting scalpel or the distal end portion of the ultrasonic scalpel to protrude through the first opening portion.

In the liquid ejecting apparatus for medical treatment of this aspect, the aperture between the outer body and the inner body has the suctioning path. The suctioning path can be commonly used with respect to different types of the scalpels. Therefore, compared to a case of having individual suctioning paths with respect to different types of the scalpels, the structure can be miniaturized.

(2) The liquid ejecting apparatus for medical treatment of the aspect described above may be configured such that the liquid ejecting apparatus for medical treatment further includes a controller that controls the liquid ejecting scalpel and the ultrasonic scalpel, and a switch that is connected to the controller and instructs a beginning of use or a cessation of use for each of the scalpels, and the controller switches functions of the switch to be associated with each scalpel based on a manipulation of the manipulation section.

With the liquid ejecting apparatus for medical treatment of this aspect, a user can instruct the beginning of use or the cessation of use for each of the scalpels by the same switch before and after the switching of the scalpel to be used.

(3) The liquid ejecting apparatus for medical treatment of the aspect described above may be configured such that the manipulation section includes a slider to which the scalpels are individually connected.

With the liquid ejecting apparatus for medical treatment of this configuration, the distal end portion of the liquid ejecting scalpel or the distal end portion of the ultrasonic scalpel can be selectively protruded through the first opening portion by the slider. A user can switch the scalpel to be used through a simple manipulation.

(4) Another aspect of the invention provides a liquid ejecting apparatus for medical treatment. The liquid ejecting apparatus for medical treatment includes a liquid ejecting scalpel that ejects a liquid, an electrical scalpel that generates a high frequency current, an inner body that accommodates the liquid ejecting scalpel and the electrical scalpel and includes a first opening portion allowing a distal end portion of the liquid ejecting scalpel or a distal end portion of the electrical scalpel to protrude therethrough, an outer body that accommodates the inner body, include a second opening portion at a position corresponding to the first opening portion, and forms a suctioning path made by an aperture formed between the inner body and the outer body, and a manipulation section that is arranged in the outer body and selectively causes the distal end portion of the liquid ejecting scalpel and the distal end portion of the electrical scalpel to protrude through the first opening portion.

In the liquid ejecting apparatus for medical treatment of this aspect, the suctioning path is included in the aperture between the outer body and the inner body. Therefore, the suctioning path can be used as a common suctioning path with respect to the different types of the scalpels. Accordingly, compared to the case of having individual suctioning paths with respect to the different types of the scalpels, the structure can be miniaturized.

(5) The liquid ejecting apparatus for medical treatment of the aspect described above may be configured such that the liquid ejecting apparatus for medical treatment further includes a controller that is connected to each of the liquid ejecting scalpel and the electrical scalpel and controls the liquid ejecting scalpel and the electrical scalpel, and a switch that is electrically connected to the controller and instructs a beginning of use or a cessation of use for each of the scalpels, and the controller controls switching of functions of the switch to be associated with each scalpel based on a manipulation of the manipulation section.

With the liquid ejecting apparatus for medical treatment of this aspect, a user can instruct the beginning of use or the cessation of use for each of the scalpels by the same switch before and after the switching of the scalpel to be used.

(6) The liquid ejecting apparatus for medical treatment of the aspect described above may be configured such that the manipulation section includes the sliders to which each of the scalpels is individually connected.

With the liquid ejecting apparatus for medical treatment of this aspect, the distal end portion of the liquid ejecting scalpel or the distal end portion of the electrical scalpel can be selectively protruded through the first opening portion by the slider. A user can switch the scalpel to be used through a simple manipulation.

Not all of the multiple configuration elements included in each aspect of the invention described above are essential. In order to partially or entirely solve the above-described disadvantages, or in order to partially or entirely achieve the effects disclosed in this specification, it is possible to appropriately carry out a change, an elimination, replacement for another new configuration element, and a partial elimination of limited contents regarding a portion of the configuration elements among the plurality of configuration elements. In order to partially or entirely solve the above-described disadvantages, or in order to partially or entirely achieve the effects disclosed in this specification, it is possible to combine a portion or the entirety of the above-described technical features included in an aspect of the invention with a portion or the entirety of the above-described technical features included in another aspect of the invention so as to establish an individual aspect of the invention.

For example, an aspect of the invention can be implemented as an apparatus including one or more elements among the six elements such as the liquid ejecting scalpel, the ultrasonic scalpel, the inner body, the outer body, the suctioning path, and the manipulation section. In other words, the apparatus may have the liquid ejecting scalpel or need not have the same. The apparatus may have the ultrasonic scalpel or need not have the same. The apparatus may have the inner body or need not have the same. The apparatus may have the outer body or need not have the same. The apparatus may have the suctioning path or need not have the same. The apparatus may have the manipulation section or need not have the same.

The liquid ejecting scalpel may be configured to be as a liquid ejecting scalpel that ejects a liquid. The ultrasonic scalpel may be configured to be as an ultrasonic scalpel that generates an ultrasonic wave. The inner body may be configured to be as an inner body that accommodates the liquid ejecting scalpel and the ultrasonic scalpel and includes a first opening portion allowing the distal end portion of the liquid ejecting scalpel or the distal end portion of the ultrasonic scalpel to protrude therethrough. The outer body may be configured to be as an outer body that accommodates the inner body and includes the second opening portion at a position corresponding to the first opening portion. The suctioning path may be configured to be as a suctioning path that is made by an aperture formed between the inner body and the outer body. The manipulation section may be configured as a manipulation section that is arranged in the outer body and selectively causes the distal end portion of the liquid ejecting scalpel or the distal end portion of the ultrasonic scalpel to protrude through the first opening portion.

Such an apparatus can be implemented as a liquid ejecting apparatus for medical treatment as well as it can be used as another apparatus other than the liquid ejecting apparatus for medical treatment. According to the aspects described above, it is possible to solve at least one of various problems such as miniaturization of the apparatus, reduction in cost, saving of resources, facilitation in manufacturing, and enhancement of usability. A portion or the entirety of the above-described technical features according to each aspect of the liquid ejecting apparatus can be applied to the apparatus.

The invention can be implemented in various forms other than the instruments. For example, it is possible to be implemented in forms of a scalpel for medical treatment, a scalpel switching apparatus for medical treatment, a scalpel switching method for medical treatment, a liquid ejecting method, and a medical instrument.

### BRIEF DESCRIPTION OF THE DRAWINGS

The invention will be described with reference to the accompanying drawings, wherein like numbers reference like elements.

FIG. 1 is a diagram illustrating a configuration of a scalpel apparatus for medical treatment.

FIG. 2 is a schematic view schematically illustrating a structure of a handpiece.

FIG. 3 is a schematic view illustrating a structure of an inner case.

FIG. 4 is a diagram illustrating a relationship between a manipulation section and a controller.

FIG. 5 is a diagram illustrating another handpiece.

FIG. 6 is a schematic view schematically illustrating a configuration of a functional portion accommodation case.

FIG. 7 is a schematic view schematically illustrating a configuration of another functional portion accommodation case.

FIG. 8 is a schematic view illustrating a state where a probe moves.

FIG. 9 is a diagram illustrating a guide member.

### DESCRIPTION OF EXEMPLARY EMBODIMENTS

### A. First Embodiment

### A1. Scalpel Apparatus for Medical Treatment

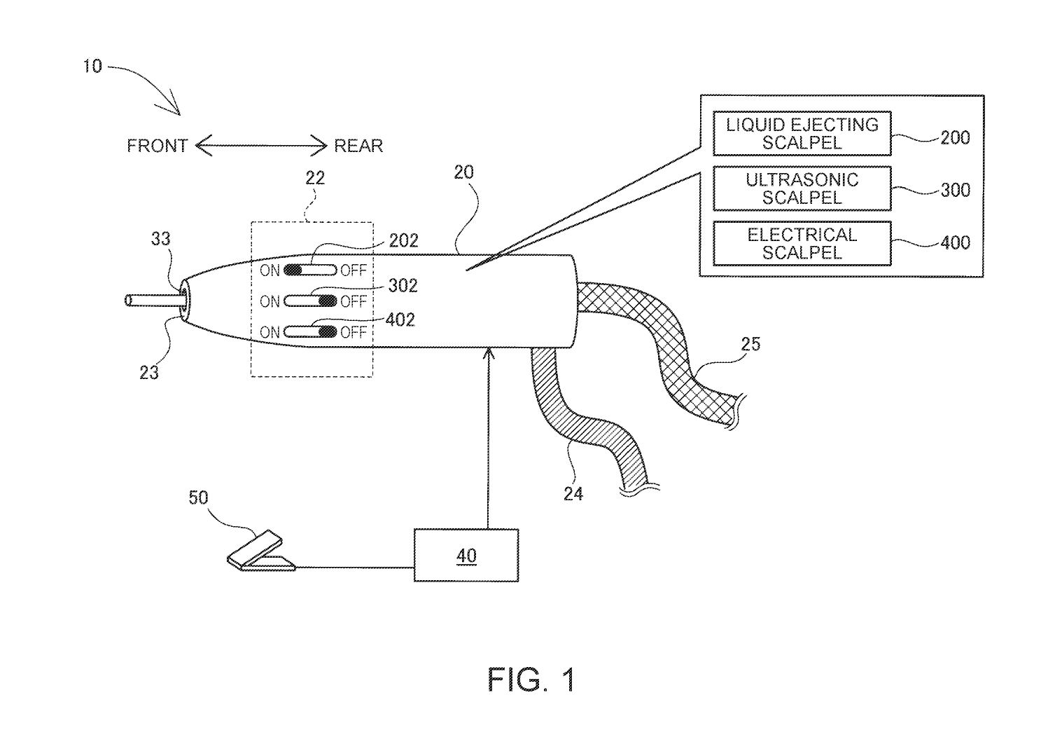

FIG. 1 is a diagram illustrating a configuration of a scalpel apparatus 10 for medical treatment as a first embodiment of the invention. The scalpel apparatus 10 for medical treatment is a medical instrument including multiple types of scalpels for medical treatment.

The scalpel apparatus 10 for medical treatment includes a handpiece 20 and a controller 40. The handpiece 20 accommodates a liquid ejecting scalpel 200, an ultrasonic scalpel 300, and an electrical scalpel 400 inside thereof.

The liquid ejecting scalpel 200 is a surgical instrument which ejects a liquid to perform incision or excision of a lesion site by a stream flow. The ultrasonic scalpel 300 is a surgical instrument which vibrates a vibrator using an ultrasonic frequency and emulsifies or destroys a biological tissue by the vibration to perform incision or excision of a lesion site. The electrical scalpel 400 is a surgical instrument which performs incision or coagulation (hemostasis) of a lesion site by a thermic effect of a high frequency current.

The handpiece 20 includes a manipulation section 22. The manipulation section 22 includes sliders 202, 302, and 402. A user of the scalpel apparatus 10 for medical treatment selects one slider among the three sliders included in the manipulation section 22 and operates the same so that a scalpel to be used can be selected from the three scalpels accommodated in the handpiece 20. The slider 202 is a slider to select the liquid ejecting scalpel 200. The slider 302 is a slider to select the ultrasonic scalpel 300. The slider 402 is a slider to select the electrical scalpel 400. When using each of the scalpels, the user causes the slider corresponding to the scalpel to be used to be turned ON. While one slider is turned ON, the remaining sliders are turned OFF.

When a user selects and operates the slider, a distal end portion of the scalpel corresponding to the selected slider protrudes through an opening portion 33 formed in the handpiece 20. The user performs incision, excision, hemostasis, and the like of a lesion site by bringing the distal end portion of the scalpel protruding through the opening portion 33 to be in contact or causing the distal end portion thereof to approach the lesion site.

A suctioning path 23 is formed outside the opening portion 33. Inclusions such as excisions, blood, and a waste liquid which lie in the surrounding of the opening portion 33 are subjected to suctioning through the suctioning path 23. The suctioning path 23 will be described later in detail.

A cable-shaped member group 25 extends from the rear end of the handpiece 20. The cable-shaped member group 25 is a bundle of pipes and electrical wires necessary to operate the three scalpels.

The controller 40 is connected to the liquid ejecting scalpel 200, the ultrasonic scalpel 300, and the electrical scalpel 400. The controller 40 controls the operations of the three scalpels. A foot switch 50 is connected to the controller 40. When a user turns ON/OFF the foot switch 50, the scalpel selected by the user through the manipulation section 22 is driven.

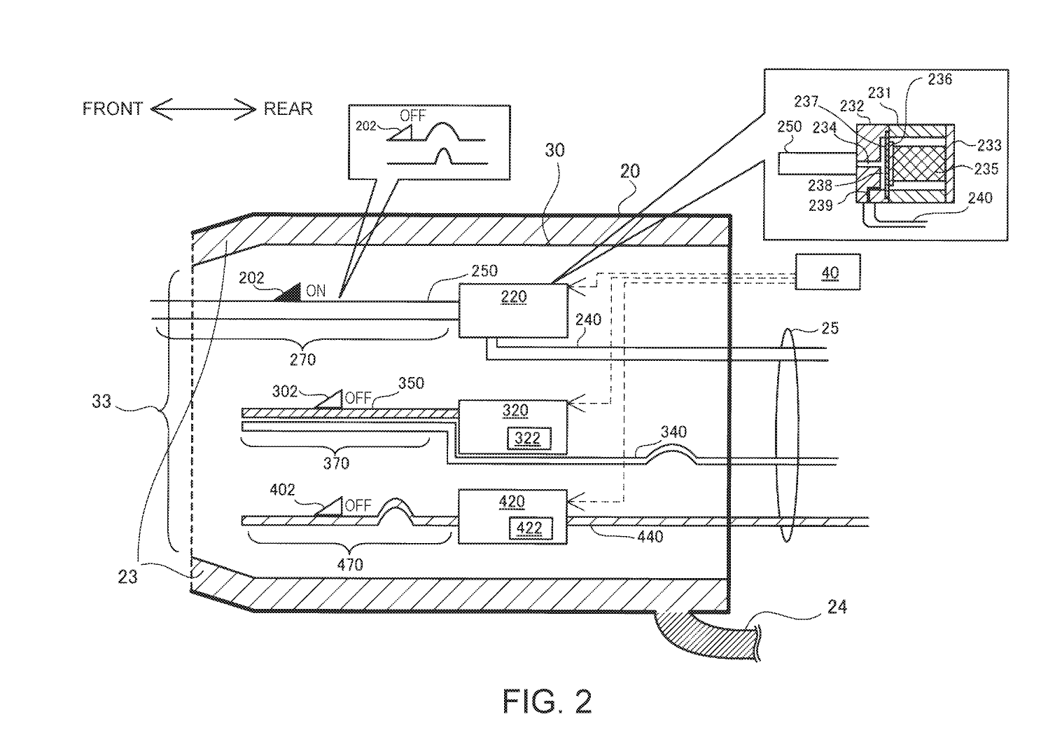

FIG. 2 is a schematic view schematically illustrating a structure of the handpiece 20. The handpiece 20 accommodates an inner case 30 therein. The liquid ejecting scalpel 200, the ultrasonic scalpel 300, and the electrical scalpel 400 are accommodated in the inner case 30.

The liquid ejecting scalpel 200 includes an actuator 220, a liquid supply channel 240, and a liquid ejection channel 250. The liquid supply channel 240 is a flow channel to supply a liquid pumped from a pump (not illustrated) which is prepared outside the handpiece 20, to the actuator 220. The liquid supply channel 240 is composed of a member having flexibility. In the embodiment, the liquid supply channel 240 is formed with a PEEK member. The liquid supply channel 240 may be formed with various flexible members such as polyvinyl chloride, silicon, and thermoplastic elastomers. As the liquid to be supplied to the actuator 220, it is possible to employ various liquids such as sterile water for medical use or pure water.

The actuator 220 applies a pulsation to a liquid which is supplied from the liquid supply channel 240. The liquid applied with a pulsating flow is supplied to the liquid ejection channel 250, thereby being ejected from a distal end of the liquid ejection channel 250 as a pulsatile liquid. The pulsatile liquid denotes a liquid in a state where a flow rate or a flow velocity is accompanied by fluctuation. As a form of ejecting a liquid in a pulsatile manner, intermittent ejection in which liquid is ejected while repeating the ejection and a pause is included. However, it is acceptable as long as the flow rate or the flow velocity of a liquid fluctuates, and thus, it does not necessarily have to be the intermittent ejection.

As illustrated in the drawing, the actuator 220 includes a first case 231, a second case 232, a third case 233, a piezoelectric element 235, a reinforcement plate 236, and a diaphragm 237. The first case 231 is a tubular member. An end of the first case 231 is connected to the second case 232. Another end of the first case 231 is sealed by the third case 233. The piezoelectric element 235 is arranged in a space formed inside the first case 231.

The piezoelectric element 235 is a laminated piezoelectric element. One end of the piezoelectric element 235 is fixed to the diaphragm 237 via the reinforcement plate 236. Another end of the piezoelectric element 235 is fixed to the third case 233. The diaphragm 237 is formed with a thin metal film and a peripheral edge thereof is fixed to the first case 231. An accommodation chamber 238 is formed between the diaphragm 237 and the second case 232. The volume of the accommodation chamber 238 changes in response to driving of the piezoelectric element 235.

A first flow channel 239 which allows a liquid to flow into the accommodation chamber 238 is formed in the second case 232. The first flow channel 239 is connected to the liquid supply channel 240. A second flow channel 234 which allows a liquid accommodated in the accommodation chamber 238 to flow out is formed in the second case 232. The second flow channel 234 is connected to the liquid ejection channel 250.

A drive signal at a predetermined frequency is applied from the controller 40 to the piezoelectric element 235. The piezoelectric element 235 vibrates at a predetermined frequency upon reception of the drive signal from the controller 40. When the piezoelectric element 235 vibrates, the volume of the accommodation chamber 238 changes via the diaphragm 237, and then, the liquid accommodated in the accommodation chamber 238 is pressurized. A pulsation is applied to the liquid which is compressed or decompressed at a predetermined frequency. The liquid passes through the second flow channel 234 and the liquid ejection channel 250 and is ejected to the outside as the pulsatile liquid. The actuator 220 has such a configuration.

The slider 202 is connected to the liquid ejection channel 250. When the slider 202 is slid to be turned ON, the liquid ejection channel 250 moves in a sliding direction. Then, the distal end portion of the liquid ejection channel 250 protrudes through the opening portion 33 formed in the distal end portion of the inner case 30. The actuator 220 is fixed to the inner case 30. Accordingly, even though the slider 202 moves, the actuator 220 does not move. The liquid ejection channel 250 is also referred to as a probe 270 for convenience of description.

The probe 270 has a sufficient length to be movable by the sliding of the slider 202. When the slider 202 is turned OFF, the probe 270 is accommodated in the inner case 30 in a flexed state. The liquid ejection channel 250 is composed of a member having flexibility. In the embodiment, the liquid ejection channel 250 is formed with a PEEK member. The liquid ejection channel 250 may be formed with various flexible members such as polyvinyl chloride, silicon, and thermoplastic elastomers. The liquid ejection channel 250 may be configured of a member having elasticity. The liquid ejecting scalpel 200 has such a configuration.

The ultrasonic scalpel 300 includes an actuator 320, an ultrasonic wave transmission body 330, a liquid supply channel 340, and a liquid ejection channel 350. A configuration including the ultrasonic wave transmission body 330 and the liquid ejection channel 350 at a distal end from the actuator 320 is also referred to as a probe 370.

The actuator 320 includes a vibrator 322 that generates an ultrasonic wave. The vibrator 322 is connected to the controller 40. A drive signal having a predetermined voltage is applied to the vibrator 322 from the controller 40. The vibrator 322 generates a vibrating ultrasonic wave upon reception of the drive signal. The ultrasonic wave generated by the vibrator 322 transmits the ultrasonic wave transmission body 330, and destroys or emulsifies a biological tissue existing at a distal end of the ultrasonic wave transmission body 330.

The liquid supply channel 340 is a flow channel to supply a liquid pumped from the pump which is prepared outside the handpiece 20, to the distal end of the probe 270. The liquid supplied from the liquid supply channel 340 transmits an ultrasonic wave which is transmitted to the ultrasonic wave transmission body 330, to a lesion site. The liquid supplied from the liquid supply channel 340 performs washing of the lesion site. As the liquid, it is possible to employ various liquids such as sterile water for medical use or pure water. In the embodiment, the liquid supply channel 240 is formed with the PEEK member. The liquid supply channel 240 may be formed with various resins such as polyvinyl chloride, silicon, and thermoplastic elastomers.

In the embodiment, the probe 370 may have a configuration in which the ultrasonic wave transmission body 330 and the liquid ejection channel 350 come into contact with each other outside to be arranged in parallel. The slider 302 is connected to the probe 370. When the slider 302 is slid to be turned ON, the probe 370 and the actuator 320 move in the sliding direction. Then, a distal end portion of the probe 370 protrudes through the opening portion 33.

The liquid supply channel 340 has a sufficient length to be movable by the sliding of the slider 302 at the rear from the actuator 320. The liquid supply channel 340 is composed of a member having flexibility. In the embodiment, the liquid supply channel 340 is formed with a PEEK member. The liquid supply channel 340 may be formed with various flexible members such as polyvinyl chloride, silicon, and thermoplastic elastomers. The liquid supply channel 340 may be configured of a member having elasticity. When the slider 302 is turned OFF, the liquid supply channel 340 is accommodated in the inner case 30 in a flexed state at the rear of the actuator 320. The ultrasonic scalpel 300 has such a configuration.

The electrical scalpel 400 includes an actuator 420, an electrical cable 440, and a high frequency treatment electrode 450. The high frequency treatment electrode 450 is also referred to as a probe 470, for convenience of the description.

The electrical cable 440 is a cable that supplies electrical power to the actuator 420. The electrical cable 440 is connected to a high frequency current generator (not illustrated) outside the handpiece 20. The actuator 420 supplies a high frequency current which is supplied from the electrical cable 440, to the high frequency treatment electrode 450.

The high frequency current supplied to the high frequency treatment electrode 450 flows to a lesion site. In this case, Joule heat is generated due to a load or contact resistance resulting in coagulation of protein in the lesion site, thereby making hemostasis and the like possible.

The actuator 420 includes a switching element which performs turning ON/OFF of a high frequency current to the high frequency treatment electrode 450 upon reception of the control signal from the controller 40. The actuator 420 further performs various types of controlling necessary to operate the electrical scalpel 400. The switching element may be included in the high frequency current generator. The high frequency generator may be accommodated in the actuator 420.

The slider 402 is connected to the high frequency treatment electrode 450. When the slider 402 is slid to be turned ON, the high frequency treatment electrode 450 moves in the sliding direction. Then, a distal end of the high frequency treatment electrode 450 protrudes through the opening portion 33. The actuator 420 is fixed to the inner case 30. Accordingly, even though the slider 402 moves, the actuator 420 does not move.

The high frequency treatment electrode 450 is covered by an insulating resin excluding the distal end portion. The high frequency treatment electrode 450 configuring the probe 470, and the insulating resin have flexibility. The probe 470 has a sufficient length to be movable by the sliding of the slider 402. When the slider 402 is turned OFF, the probe 470 is accommodated in the inner case 30 in a flexed state. The electrical scalpel 400 has such a configuration.

The suctioning path 23 is formed between the handpiece 20 and the inner case 30. The suctioning path 23 is connected to a suctioning pump (not illustrated) outside the handpiece 20. Inclusions such as excisions excised by each of the scalpels, and a waste liquid which lie in the surrounding of the distal end portion of the handpiece 20 are subjected to suctioning through the suctioning path 23. The suctioning path 23 is connected to a suctioning tube 24 at the rear end portion of the handpiece 20. The suctioning tube 24 is connected to the suctioning pump (not illustrated) outside the handpiece 20. A sucked substance sucked through the suctioning path 23 is sucked by the suctioning pump via the suctioning tube 24, thereby being discharged outside.

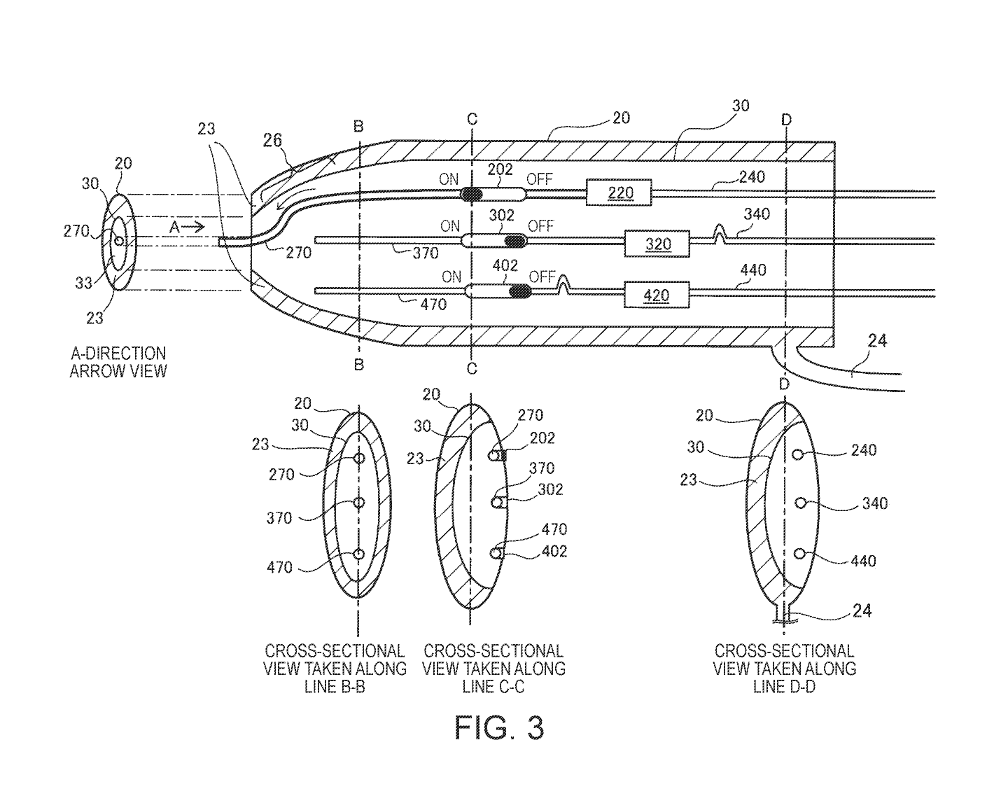

FIG. 3 is a schematic view illustrating a structure of the inner case 30. As seen in the A-direction arrow view and the cross-sectional view taken along line B-B in FIG. 3, in the vicinity of the distal end of the handpiece 20, the suctioning path 23 is formed throughout the entire periphery of the inner case 30 on the outside. As seen in the cross-sectional view taken along line C-C, a portion of an inner wall of the handpiece 20 and a portion of an outer wall of the inner case 30 are in contact with each other. The sliders 202, 302, and 402 are formed on the surface in which the handpiece 20 and the inner case 30 are in contact with each other. As seen in the cross-sectional view taken along line D-D, a portion of the inner wall of the handpiece 20 and a portion of the outer wall of the inner case 30 are in contact with each other at the rear end of the handpiece 20.

A state in which the probe moves will be described with reference to FIG. 3. The distal end portion of the inner case 30 is formed to have a curved shape. The curved portion of the inner case 30 is also referred to as a guide portion 26. When the slider of each scalpel is moved forward, the guide portion 26 guides the distal end portion of the probe of each scalpel to the opening portion 33. The probe of each scalpel moves along the guide portion 26, and every probe protrudes from the approximately same position of the opening portion 33. Since the guide portion 26 is formed to have a smoothly curved shape, there is a little friction resistance between the probe and the guide portion 26 when the probe moves along the guide portion 26, thereby allowing the probe to smoothly move.

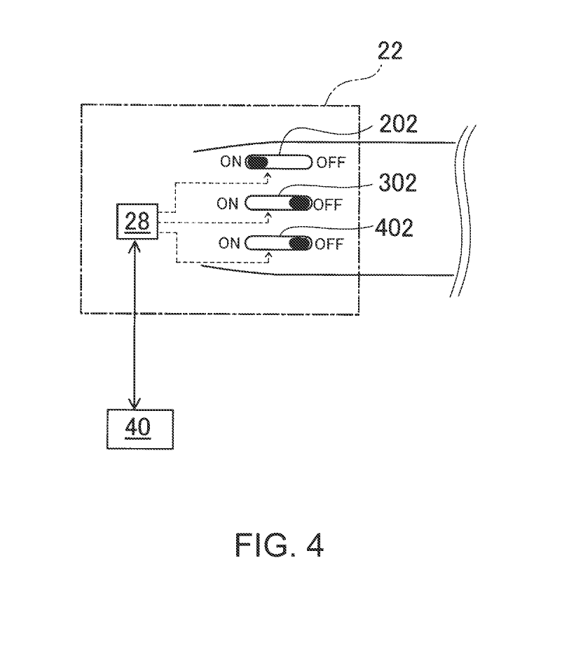

FIG. 4 is a diagram illustrating a relationship between the manipulation section 22 and the control portion 40. As illustrated in the drawing, the manipulation section 22 includes a sensor 28 that detects an ON/OFF state of each slider. The sensor 28 transmits a signal indicating the ON/OFF state of each slider to the controller 40. The controller 40 receives a signal from the sensor 28. The controller 40 controls a foot switch 50 to function as a switch to start/pause the operation of the scalpel of which the slider is turned ON, based on the ON/OFF state of each slider. A user performs the manipulation to start/pause the operation of the scalpel by using the foot switch 50 while any scalpel is used. The manipulation section 22 includes a locking function to respectively regulate only one slider among a plurality thereof to be in the ON state. The locking function allows a user to selectively use only one scalpel among the respective scalpels.

As described above, the liquid ejecting scalpel 200, the ultrasonic scalpel 300, and the electrical scalpel 400 are accommodated in the handpiece 20 (inner case 30). The handpiece 20 also includes the manipulation section 22. Therefore, the scalpel apparatus 10 for medical treatment allows a user to switch and use the each of the scalpels. Accordingly, the visual point of the user can be suppressed from moving when switching the scalpel to be used.

The liquid ejecting scalpel 200 is suitable for excising or incising a site having relatively little fibers or a site in which a minimized invasive operation is a major concern (for example, brain). The liquid ejecting scalpel 200 allows fine blood vessels or nerves to be soundly retained. The ultrasonic scalpel 300 is highly capable of destroying and emulsifying a biological tissue, thereby being suitable for excising or incising a site having relatively many fibers. The electrical scalpel is effective when performing hemostasis. Therefore, the scalpel apparatus 10 for medical treatment allows a user to use suitable scalpels, in accordance with the type of treatment.

The suctioning path 23 is formed in the aperture between the handpiece 20 and the inner case 30. The suctioning path 23 can be used as a common suctioning path with respect to the different types of the scalpels. Accordingly, compared to the case of having individual suctioning paths with respect to the different types of the scalpels, it is possible to attain miniaturization in size, reduction in weight, simplification of the structure for the handpiece 20. The suctioning path 23 is formed throughout the entire periphery of the opening portion 33 at the distal end of the handpiece 20. Accordingly, compared to the configuration in which the suctioning paths are individually provided, it is possible to widen the range in which inclusions in the surrounding of the opening portion 33 can be sucked.

Since the inner case 30 includes the guide portion 26, when each of the sliders is slid, the distal end of the probe of each scalpel protrudes from the approximately same position of the opening portion 33. Accordingly, the visual point of the user can be suppressed to be minimized from moving when switching the scalpel to be used. Therefore, poor concentration and physical exhaustion of a user during the surgical operation can be suppressed.

The controller 40 controls the foot switch 50 to function as a switch to start/pause the operation of the scalpel of which the slider is turned ON, in accordance with the ON/OFF state of each slider. Accordingly, a user can manipulate the beginning of use or the cessation of use for each of the scalpels by using the same foot switch before and after the switching of the scalpel to be used.

The slider 202 included in the liquid ejecting scalpel 200 is connected to the probe 270. When the slider 202 is slid, only the probe 270 moves, and the actuator 220 maintains a state of being fixed to the inner case 30. Accordingly, when manipulating the slider, a user can slide the slider 202 by a relatively small force. Since the actuator 220 is fixed to the inner case 30, when the slider 202 slides, movements of a centroid of the inner case 30 itself can be suppressed.

### B. Second Embodiment

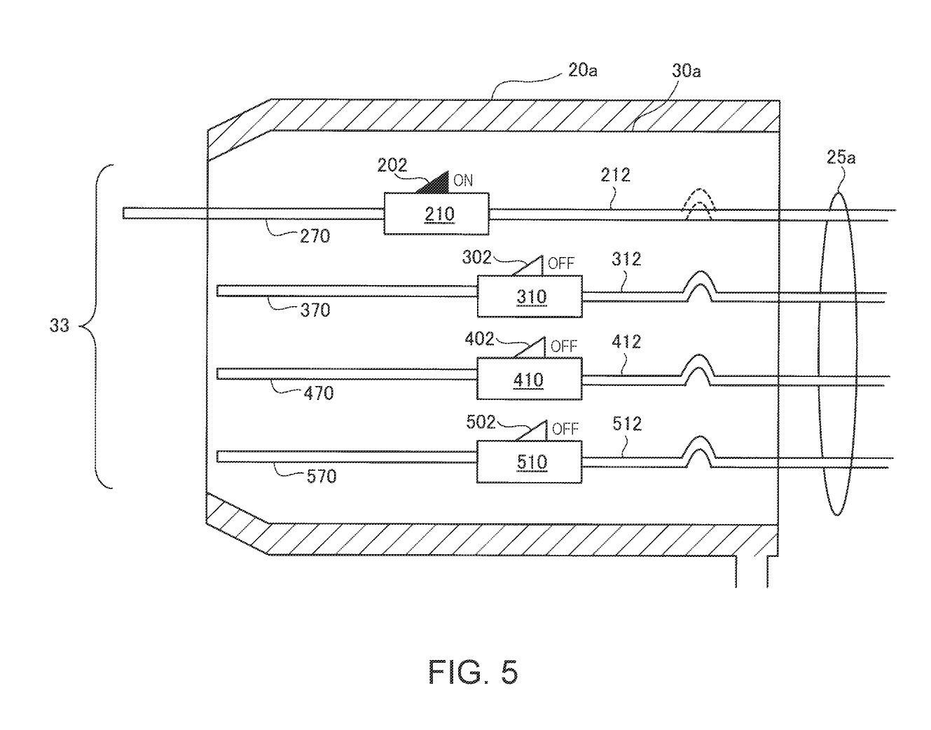

A second embodiment of the invention will be described. FIG. 5 is a diagram illustrating a handpiece 20a in the second embodiment. The difference between the second embodiment and the first embodiment is that a laser scalpel 500 is accommodated in an inner case 30a, and the scalpels are respectively accommodated in cases inside the inner case 30a. The laser scalpel 500 is a medical scalpel performing incision and excision of a biological tissue by using heat energy of a laser beam. As illustrated in the drawing, the liquid ejecting scalpel 200, the ultrasonic scalpel 300, the electrical scalpel 400, and the laser scalpel 500 are accommodated in the inner case 30a.

The liquid ejecting scalpel 200 includes a functional portion accommodation case 210. The probe 270 is stretched from the front of the functional portion accommodation case 210. A cable-shaped member group 212 is stretched from the rear of the functional portion accommodation case 210. The cable-shaped member group 212 includes the liquid supply channel 240 and a control wire through which a drive signal to drive the piezoelectric element 235 is transmitted (refer to FIG. 2). The slider 202 is connected to the functional portion accommodation case 210.

The ultrasonic scalpel 300 includes a functional portion accommodation case 310. The probe 370 is stretched from the front of the functional portion accommodation case 310. A cable-shaped member group 312 is stretched from the rear of the functional portion accommodation case 310. The cable-shaped member group 312 includes the liquid supply channel 340 (refer to FIG. 2). The slider 302 is connected to the functional portion accommodation case 310.

The electrical scalpel 400 includes a functional portion accommodation case 410. The probe 470 is stretched from the front of the functional portion accommodation case 410. A cable-shaped member group 412 is stretched from the rear of the functional portion accommodation case 410. The cable-shaped member group 412 includes the electrical cable 440 (refer to FIG. 2). The slider 402 is connected to the functional portion accommodation case 410.

The laser scalpel 500 includes a functional portion accommodation case 510. A probe 570 is stretched from the front of the functional portion accommodation case 510. A cable-shaped member group 512 is stretched from the rear of the functional portion accommodation case 510. The cable-shaped member group 512 includes an optical fiber to transmit laser light. A slider 502 is connected to the functional portion accommodation case 510.

In the embodiment, when each of the sliders is slid, each of the probes and the functional portion accommodation cases moves. When the slider of each scalpel is turned OFF, the cable-shaped member groups 212, 312, 412, and 512 are accommodated in the inner case 30a at the rear of each functional portion accommodation case in a flexed state. When the slider of each scalpel is turned ON, each of the flexed cable-shaped member groups is in an extended state. Similar to the first embodiment, when the slider of each scalpel is turned ON, each probe is guided to the opening portion 33 by a guide portion 26a included in the inner case 30a, thereby protruding through the opening portion 33.

The cable-shaped member group of each of the scalpels may include another pipe and wire in accordance with the function included in each of the scalpels, in addition to the configuration described above. The cable-shaped member group 212, the cable-shaped member group 312, the cable-shaped member group 412, and the cable-shaped member group 512 configure a cable-shaped member group 25a as a bundle. The cable-shaped member group 25a is stretched from the rear of the handpiece 20a.

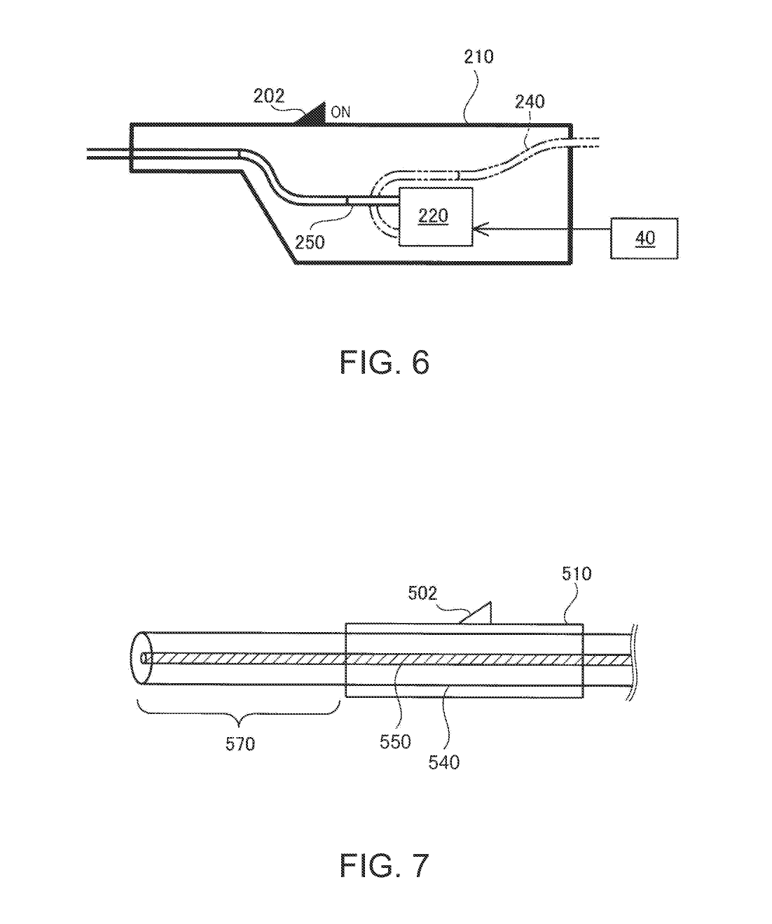

FIG. 6 is a schematic view schematically illustrating a configuration of the functional portion accommodation case 210. The actuator 220, the liquid supply channel 240, and the liquid ejection channel 250 are accommodated in the functional portion accommodation case 210. In the embodiment, the liquid supply channel 240 is connected to the front surface of the actuator 220. Regarding the function of each configuration, the description has been given in the first embodiment, thereby omitting the description.

The description for the functional portion accommodation case 310 with reference to the drawing will be omitted. The actuator 320 (FIG. 2) is accommodated in the functional portion accommodation case 310.

The description for the functional portion accommodation case 410 with reference to the drawing will be omitted. The actuator 420 (FIG. 2) is accommodated in the functional portion accommodation case 410.

FIG. 7 is a diagram schematically illustrating a configuration of the functional portion accommodation case 510. In the functional portion accommodation case 510, an optical fiber 550 through which a laser beam passes through, and a liquid supply channel 540 to which a liquid is supplied communicate with each another. The liquid supply channel 540 is inserted into the optical fiber 550.

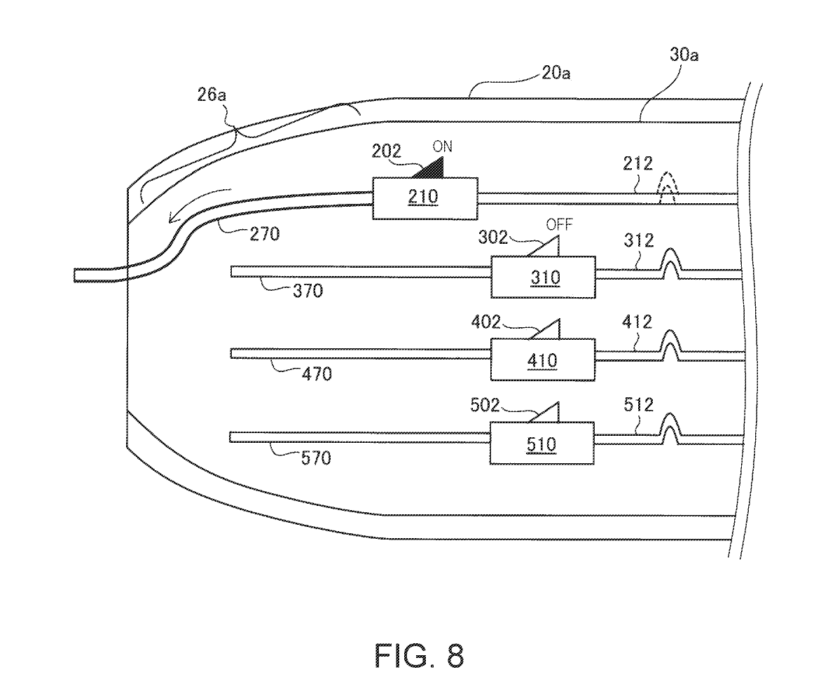

FIG. 8 is a schematic view illustrating another state where the probe moves. The inner case 30a includes the curved-shaped guide portion 26a. In the embodiment, when each of the sliders is slid, each of the functional portion accommodation cases of the scalpels moves. The guide portion 26a respectively guides the distal end portions of the probes of the scalpels to the opening portion 33 when the sliders of each of the scalpels are moved forward. The probes of the scalpels respectively move along the guide portion 26, and every probe protrudes from the approximately same position of the opening portion 33.

As illustrated above, each of the scalpels in the embodiment is individually accommodated in the cases inside the inner case 30a. Each of the sliders is connected to each of the cases. When each of the slider is slid, each of the cases and the probes moves. Accordingly, when each of the slider is OFF, the cable-shaped member group of each of the scalpels is accommodated in the inner case 30a in a flexed state. Accordingly, when each of the slider is turned OFF, it is possible to avoid the probe portion to be accommodated in a flexed state. Therefore, the distal end portion of the handpiece 20a (inner case 30a) can be thinned. Since each of the scalpels is individually accommodated in the functional portion accommodation case inside the inner case 30a, it is possible to improve the durability thereof.

### C. Modification Example

The invention is not limited to the embodiments described above and can be embodied in various aspects without departing from the scope of the invention. For example, following modifications can also be made.

### C1. Modification Example 1

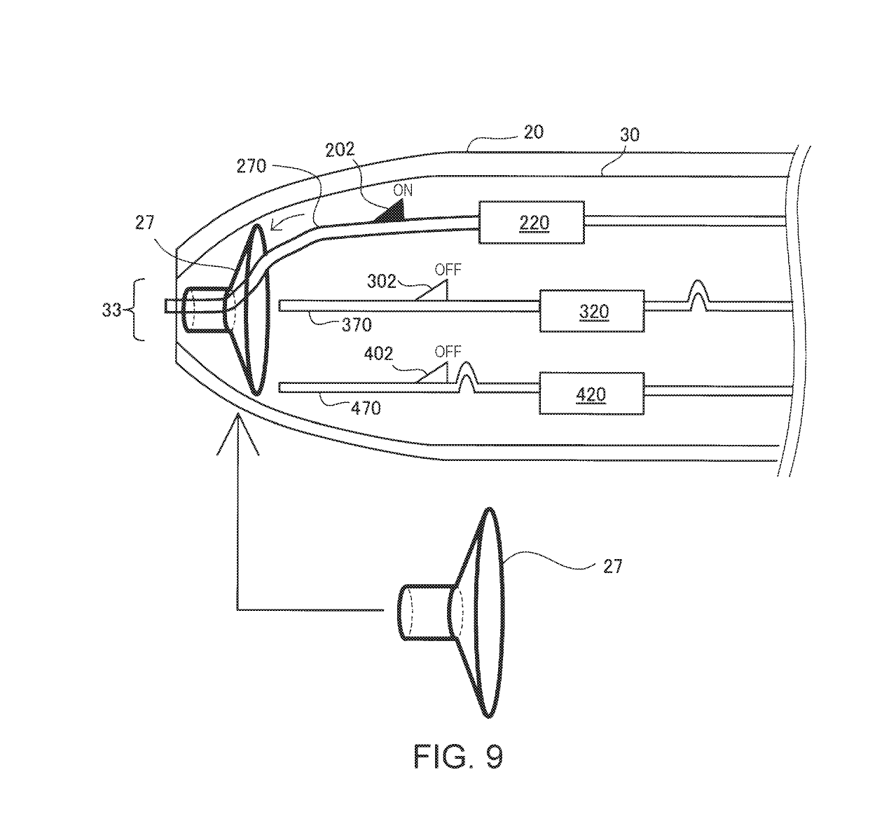

In the scalpel apparatus 10 for medical treatment, the guide portion 26 is the curved portion formed in the inner case 30. However, a guide member 27 may be separately included therein. FIG. 9 is a diagram illustrating the guide member 27. As illustrated in the drawing, the guide member 27 has a structure in which a cylindrical member 27b is connected to the center of a conical-shaped member 27a. The guide member 27 is fixed to the distal end inside the inner case 30. When the slider of each of the scalpels is slid to be in the ON state, the probe is guided from the conical-shaped member 27a to the cylindrical member 27b, thereafter, the probe is guided to the opening portion 33. The inner case 30 in a Modification Example 1 separately includes the guide member 27, and thus, there is no need to form the guide portion 26 at the distal end portion of the inner case 30. Therefore, the shapes for the distal end portion of the handpiece 20 and the distal end portion of the inner case 30 are not limited. For example, a user can form the distal ends of the handpiece 20 and the inner case 30 to have a shape so as to be easily grasped.

### C2. Modification Example 2

The ultrasonic scalpel 300 may be configured not to have the liquid ejecting channel 350. It is possible to attain the miniaturization of the handpiece 20 (inner case 30).

### C3. Modification Example 3

In the scalpel for medical treatment as a liquid ejecting apparatus for medical treatment of the embodiments, a user can switch and use each of the scalpels by the manipulation section as described above. In other words, the scalpel apparatus 10 for medical treatment can be used by the following usage method.

There is provided a method of using a liquid ejecting apparatus for medical treatment including a liquid ejecting scalpel that includes a liquid ejecting portion ejecting a liquid and a pulsating flow applier applying a pulsating flow to the liquid, an ultrasonic scalpel that has an ultrasonic wave generation portion generating a ultrasonic wave, an electrical scalpel provided with a high frequency treatment electrode, an inner body that accommodates the liquid ejecting scalpel, the ultrasonic scalpel, and the electrical scalpel and includes a first opening portion through which a distal end portion of the liquid ejecting scalpel, a distal end portion of the ultrasonic scalpel, or a distal end portion of the electrical scalpel can protrude, an outer body that accommodates the inner body, includes a second opening portion at a position corresponding to the first opening portion, and forms a suctioning path made by an aperture formed between the inner body and the outer body, and a manipulation section that is arranged in the outer body and selectively causes the distal end portion of the liquid ejecting scalpel and the distal end portion of the ultrasonic scalpel to protrude through the first opening portion.

The method of using a liquid ejecting apparatus for medical treatment includes at least using one scalpel, switching from one scalpel to another scalpel by manipulating the manipulation section, and using another scalpel.

According to the method of using a liquid ejecting apparatus for medical treatment, a user using the liquid ejecting apparatus for medical treatment can be suppressed in the visual point oriented to the distal end of the scalpel from being moving when switching the scalpel to be used.

### C4. Modification Example 4

In addition to Modification Example 3, the liquid ejecting apparatus for medical treatment can be used as follows.

There is provided the method of using a liquid ejecting apparatus for medical treatment disclosed in Modification Example 3. The liquid ejecting apparatus for medical treatment further includes a controller that controls each of the scalpels, and a switch that instructs a beginning of use and a cessation of use for each of the scalpels. The liquid ejecting apparatus for medical treatment includes selecting one scalpel by manipulating the manipulation section, beginning use of one scalpel by manipulating the switch, ceasing use of one scalpel by manipulating the switch, selecting another scalpel by manipulating the manipulation section, beginning use of another scalpel by manipulating the switch, ceasing use of another scalpel by manipulating the switch.

According to the method of using the liquid ejecting apparatus for medical treatment, a user can instruct the beginning of use or the cessation of use for each of the scalpels by using the same switch before and after the switching of the scalpel to be used.

What is claimed is:

1. A liquid ejecting apparatus for medical treatment comprising:
a liquid ejecting scalpel that ejects a liquid;
an ultrasonic scalpel that generates an ultrasonic wave;
an inner body that accommodates the liquid ejecting scalpel and the ultrasonic scalpel and includes a first opening portion allowing a distal end portion of the liquid ejecting scalpel or a distal end portion of the ultrasonic scalpel to protrude therethrough;
an outer body that accommodates the inner body and includes a second opening portion at a position corresponding to the first opening;
a suctioning path that is made by an aperture formed between the inner body and the outer body; and
a manipulation section that is arranged in the outer body and selectively causes the distal end portion of the liquid ejecting scalpel or the distal end portion of the ultrasonic scalpel to protrude through the first opening portion.

2. The liquid ejecting apparatus for medical treatment according to claim 1, further comprising:
a controller that controls the liquid ejecting scalpel and the ultrasonic scalpel; and
a switch that is connected to the controller and instructs a beginning of use or a cessation of use for each of the scalpels,
wherein the controller switches functions of the switch to be associated with each scalpel based on a manipulation of the manipulation section.

3. The liquid ejecting apparatus for medical treatment according to claim 1,
wherein the manipulation section includes a slider to which the scalpels are individually connected.

4. A liquid ejecting apparatus for medical treatment comprising:
a liquid ejecting scalpel that ejects a liquid;
an electrical scalpel that generates a high frequency current;
an inner body that accommodates the liquid ejecting scalpel and the electrical scalpel and includes a first opening portion allowing a distal end portion of the liquid ejecting scalpel or a distal end portion of the electrical scalpel to protrude therethrough;
an outer body that accommodates the inner body, includes a second opening portion at a position corresponding to the first opening portion, and forms a suctioning path made by an aperture formed between the inner body and the outer body; and
a manipulation section that is arranged in the outer body and selectively causes the distal end portion of the liquid ejecting scalpel and the distal end portion of the electrical scalpel to protrude through the first opening portion.

5. The liquid ejecting apparatus for medical treatment according to claim 4, further comprising:
a controller that controls the liquid ejecting scalpel and the electrical scalpel; and
a switch that is connected to the controller and instructs a beginning of use or a cessation of use for each of the scalpels,
wherein the controller switches functions of the switch to be associated with each scalpel based on a manipulation of the manipulation section.

6. The liquid ejecting apparatus for medical treatment according to claim 4,
wherein the manipulation section includes the sliders to which the scalpels are respectively connected one on one.
